(19) United States
(12) Patent Application Publication
(10) Pub. No.: US 2013/0274171 A1
(43) Pub. Date: Oct. 17, 2013

(54) TREFOIL FACTORS (TFF) FOR THE TREATMENT OF CHRONIC PULMONARY DISEASES

(71) Applicants: Kaare Fiala, Frederiksberg (DK); Lars Heslet, Gentofte (DK)

(72) Inventors: Kaare Fiala, Frederiksberg (DK); Lars Heslet, Gentofte (DK)

(73) Assignee: TRIFOILIUM APS, Hoersholm (DK)

(21) Appl. No.: 13/701,582

(22) PCT Filed: Jun. 3, 2011
(86) PCT No.: PCT/DK2011/050197

§ 371 Date: Jan. 30, 2013

(30) Foreign Application Priority Data
Jun. 4, 2010 (DK) PA 2010 70247

(51) Int. Cl. A61K 38/17

(52) U.S. Cl. CPC A61K 38/1709
USPC 514/1.1; 530/350

(57) ABSTRACT

The present invention provides methods for enhancing or inducing airway epithelial repair and/or regeneration and/or for the normalization of airway epithelial remodeling, using local deposition of one or more pro-regenerative factors, preferably a peptide or protein from the trefoil family factor (TFF) family. The pro-regenerative factor is administered to a patient as a treatment of Chronic obstructive pulmonary disease (COPD), chronic bronchitis, Cystic fibrosis, Asthma, Bronchiectasis, Panbronchiolitis, pneumonia, ventilator associated pneumonia (VAP), hospital acquired pneumonia (HAP), community acquired pneumonia (CAP), severe community acquired pneumonia (sCAP) and pneumocystis carinii or to patients suffering from lung damage reducing the function of the mucociliary transport system based on fully functioning cilia secondary to, e.g. inhalation of toxic fumes. The present invention also provide therapy and or prophylactic or preemptive, i.e. prophylaxis based on a predefined group of patients like intervention in smokers in order to enhance ongoing airway epithelial repair and/or regeneration of the airways cilia improving the mucociliary clearance. The pro-regenerative factor is administered intratracheal, intrabronchial, intraalveolar or broncioalveolar. Administration for example via inhalation of an aerosol or as a dry powder and/or administered as a bronchoalveolar lavage (BAL) an effective amount of the agent.

All patent and non-patent references cited in the application, or in the present application, are also hereby incorporated by reference in their entirety.

### FIELD OF INVENTION

The present invention provides a method the enhancement of airway epithelial repair and/or regeneration of the mucociliary clearance mechanism and/or the normalization of airway epithelial remodeling in a subject suffering from, for example Asthma, Chronic obstructive pulmonary disease (COPD), Chronic bronchitis, Smokers Lung, Cystic fibrosis, Bronchiectasis, Panbronchiolitis, Lung cancer, Pneumonia, Ventilator associated pneumonia (VAP), Hospital acquired pneumonia (HAP), Community acquired pneumonia (CAP), Severe community acquired pneumonia (sCAP) or Pneumocystis carinii by administering to the subject an effective amount of one or more pro-regenerative factor, preferably a TFF member and/or a TFF dimer, more preferably TFF3 or TFF 3 dimer via pulmonary airway administration.

### BACKGROUND OF INVENTION

The Respiratory epithelium is found lining the upper and lower respiratory tracts, where it serves to moisten and protect the airways by the so-called mucociliary clearance. It also functions as a barrier to potential pathogens and foreign objects, preventing infection by action of the ciliary escalator (described herein below).

The respiratory epithelium is known as ciliated pseudostratified columnar epithelium due to the arrangement of the columnar epithelial cells; the nuclei are not aligned in the same plane and make it appear as though several layers of cells are present. Actually, all cells make contact with the basement membrane and are therefore a single layer of cells; hence the epithelium is called pseudostratified. Goblet cells are present amongst the columnar cells and secrete mucus which keeps the epithelium moist and traps particulate material moving through the airway.

Certain parts of the respiratory tract, such as the oropharynx, are subject to the abrasive swallowing of food. To prevent the destruction of the respiratory epithelium in these areas it changes to stratified squamous epithelium which is better suited to the constant sloughing and abrasion. The squamous layer of the oropharynx is continuous with the esophagus.

The cilia of the respiratory epithelium beat in a concerted effort to move secreted mucus containing trapped foreign particles and cellular debris, dead cells, like dead alveolar macrophages towards the direction to oropharynx. The narrowest part of the airways is located at the central airways where the coughing mechanism produces sufficient air velocity to enhance turbulence with sufficient sheer stress to tear sputum away form the tracheal/main bronchial wall, to expectorate or swallow sputum to the stomach where the acidic pH inactivates foreign material and micro-organisms. The airway system from the small airways to the central airways, where the coughing mechanism becomes effective, is collectively known as the ciliary escalator and serves two functions: to clear the distal respiratory tract from bacteria foreign material dead cells for the alveoli, and to prevent mucus accumulation in the lungs in order to secure a normal gas exchange. The Ciliary function is thus a crucial part of the airway host defense.

The mucus layer on top of the ciliae is biphasic with a serous sol layer in which the cilia beat upwards and more central toward the airway lumen is the viscoelastic or gel layer

The epithelial cell lining of the airways is involved in the protection of the airway mucosa from major sources of injury including inhaled toxic particles such as tobacco smoke, pollutants, fumes and microbiological agents like bacteria and virus. And further to transport dead alveolar cells, cellular debris from the periphery and upwards to the trachea, which unless cleared from the alveolar region liberates their content of proteases like elastase, destroying the lung tissue with and end-stage chronic obstructive lung disease and emphysema. The epithelial lining of the airways provides an efficient barrier against microorganisms and aggressive molecules through interdependent functions including the following: mechanical clearance of the mucus; homeostasis of ion and water transport; biochemical antibacterial, antioxidant, and antiprotease functions; and a cellular barrier function by means of intercellular epithelial junctions. All these functions are fundamental to the protection and maintenance of the integrity of the airway epithelium.

The airway epithelium may be rapidly disturbed after any infectious or inflammatory-related in chronic diseases such as asthma, chronic obstructive pulmonary disease (COPD), bronchietasis and cystic fibrosis.

The epithelium of the airways is frequently injured by inhaled toxic particles such as tobacco smoke, pollutants, fumes and microbiological agents like bacteria and virus, and further from the lung periphery and upwards, dead alveolar cells and cell debris rich in proteases. Subsequent to such injuries a wound healing process involving epithelial repair, regeneration and remodelling is initiated with a specific ordered sequence of events:
1. Early repair phase of the epithelial barrier function consists of migration of neighboring cells. Within approximately 24 hours, many of these cells enter the cell cycle.
2. The second repair phase is characterized by inflammatory processes, cell proliferation and differentiation occurs leading to cytological and histological changes in the airway structure over time; a process which is called airway remodeling.

In chronic obstructive pulmonary disease (COPD), exacerbations are generally associated with several causes, including cigarette smoking, exposure to cigarette smoke, and exposure to environmental pollution, especially particulates pollutants, viruses and bacteria. These conditions are responsible for an airway inflammation with excess of inflammatory mediators, and proinflammatory cytokines released by activated epithelial and inflammatory cells. The normal response of the airway surface epithelium to injury includes a succession of cellular events, with a whole spectrum from the loss of the surface epithelium integrity to only partial shedding of the epithelium or even complete denudation of the basement membrane.

Pro-regenerative factors enhance epithelial repair with or without improved clearance function both in respect to central airways and most importantly from peripheral airways and alveoli, with airway restitution therapeutic potential could particularly include both a quantitative and qualitative change in sputum, i.e. reduction of the mucus production by suppression of transformation of cells into goblet cells with less viscid mucous properties. This in turn may improve mucus rheology with a less thick mucous production into a more serous production being less viscous.

Several pro-regenerative factors of the lungs are known and include defense proteins such as defensin; surfactant proteins such as SP-A, SP-B, SP-D; mucins; sIgA, lactoferrin, glycosaminoglycans, Clara cell secretory protein (CCSP), tight junction proteins such as Z01, occludin, claudins and Hepatocyte growth factor (HGF).

Cellular and molecular factors involved in airway repair and regeneration are modulated by Matrix metalloproteinases (MMPs), cytokines and growth factors released by epithelial and mesenchymal cells.

The trefoil factor family of proteins are characterised by a 40-amino acid trefoil motif that contains 3 conserved disulfide bonds. The 3 intrachain disulfide bonds form the trefoil motif (TFF domain). The trefoil motif is known in the art, e.g. Taupin and Podolsky, Nat Rev Mol Cell Bio. 4(9):721-32, 2003; Hoffmann et al., Histol Histopathol 16(I):319-34, 2001; and Thim, Cell Mol Life Sci 53(II-12):888-903, 1997.

In humans, three distinct members of the trefoil peptides have been identified so far. TFF1 or pS2 was first detected in a mammary cancer cell line as an estrogen-inducible gene. In human stomach, it is predominantly located in the foveolar cells of the gastric mucosa. TFF2 (formerly spasmolytic polypeptide or SP) was first purified from porcine pancreas and is expressed in mucous neck cells, deep pyloric glands, and Brunner's glands. TFF3 or intestinal trefoil factor (ITF) was the last to be identified and is predominantly expressed in the goblet cells of the small and large intestine. The trefoil peptides are involved in mucosal healing processes and are expressed at abnormal elevated levels in neoplastic diseases. A wide range of human carcinomas and gastrointestinal inflammatory malignancies, including peptic ulceration and colitis, Crohn's syndrome, pancreatitis, and biliary disease, aberrantly express trefoil peptides. Orthologues of these human proteins have been identified in other animals; for example, rats, mice and primates.

TFFs are stable secretory proteins expressed in gastrointestinal mucosa. Their functions are not defined.

The three trefoil family member genes; trefoil family factor I (TFF1), trefoil family factor II (TFF2) and trefoil family factor III (TFF3) are found in a cluster on chromosome 21.

### SUMMARY OF INVENTION

A first aspect of the invention is to provide a composition comprising an effective amount of one or more pro-regenerative factors for use as a medicament for pulmonary administration in a subject for the enhancement of airway epithelial repair and/or regeneration and/or the normalization of airway epithelial remodeling.

Another aspect of the present invention relates to the use of a composition according to claims 1 through 18 for the enhancement of airway epithelial repair and/or regeneration and/or the inhibition of airway epithelial remodeling via pulmonary administration in a subject.

Yet another aspect of the present invention relates to the use of a composition comprising an effective amount of one or more pro-regenerative factors as the active ingredient for the manufacture of a medicament for pulmonary administration in a subject for the enhancement of airway epithelial repair and/or regeneration and/or the normalization of airway epithelial remodeling.

A further aspect of the invention is to provide a composition comprising one or more pro-regenerative factors as the active ingredient for use as a medicament for pulmonary administration in a subject for the enhancement of airway epithelial repair and/or regeneration and/or the normalization of airway epithelial remodeling.

Another aspect of the present invention relates to a method for enhancing airway epithelial repair and/or regeneration and/or for the normalization of airway epithelial remodeling in a human subject comprising administering an effective amount of one or more pro-regenerative factors to a subject via pulmonary administration.

When used herein the term “deposition of drugs in the lungs” and other variations on this theme is meant to describe local pulmonary drug administration via the airways and not systemic drug administration as visualized in FIG. 2.

For purposes of the present invention by “the enhancement of airway epithelial repair and/or regeneration and/or the normalization of airway epithelial remodeling” it is meant any detectable improvement in the repair and/or regeneration of the airway epithelial lining and/or normalization of airway epithelial remodeling, the methods for which are described herein below.

### DEFINITIONS AND ABBREVIATIONS

Amino Acid Residue: That part of the amino acid which is present in the polypeptide chain in which the amino acid is linked to other amino acids by peptide (amide) bonds. The amino acid residues described herein are preferably in the “L” isomeric form. However, the amino acid encompasses every amino acid such as L-amino acid, D-amino acid, alpha-amino acid, beta-amino acid, gamma-amino acid, natural amino acid and synthetic amino acid or the like, as long as the desired functional property is retained by the polypeptide. Further included are natural or synthetic amino acids which have been modified. NH₂ refers to the free amino group present at the amino terminus of a polypeptide. COOH refers to the free carboxy group present at the carboxy terminus of a polypeptide. Standard polypeptide abbreviations for amino acid residues are used herein.

It should be noted that all amino acid residue sequences represented herein by formulae have a left-to-right orientation in the conventional direction of amino terminus to carboxy terminus. Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

Modified amino acid: an amino acid wherein an arbitrary group thereof is chemically modified. In particular, a modified amino acid chemically modified at the alpha-carbon atom in an alpha-amino acid is preferable.

Polypeptide: The phrase polypeptide refers to a molecule comprising amino acid residues which do not contain linkages other than amide linkages between adjacent amino acid residues. The phrase peptide is used accordingly.

Pulmonary deposition of drug: When used herein the term “deposition of drugs in the lungs” and other variations on this theme is meant to describe local pulmonary drug administration and not systemic drug administration via the airways as visualized in FIG. 2. Pulmonary deposition or delivery of a local dose thus refers to topical administration to the lung for diseases of the lungs. Alternatively, a systemic dose typically describes administration via the lung for absorption from the alveolar region to the circulation to treat systemic disorders, such as diabetes, migraine, osteoporosis, and hormone regulation

When used herein “airway epithelium” “respiratory epithelium” and “pulmonary epithelium” are meant to be used interchangeably.

According to the present invention, a pharmaceutically effective amount or a therapeutically effective amount is to be understood as an amount sufficient to induce a desired biological result i.e. the effective dose to be determined by a qualified practitioner who may titrate dosages to achieve the desired response. Factors for consideration of dose will include potency, bioavailability, desired pharmacokinetic/pharmacodynamic profiles, and condition of treatment, patient-related factors such as weight, health, age etc. or other factors known to the practitioner.

### DESCRIPTION OF DRAWINGS

FIG. 1.A) A sequence alignment of human TFF protein 1-3. B) An alignment of the protein sequences of human TFF1, human TFF2, human TFF3, mouse TFF1, mouse TFF2, mouse TFF3, rat TFF1, rat TFF1 and pig TFF2. Identical residues are marked “*”, residues with conservative substitutions are marked “:”, and semi-conservative substitutions are marked with “.”.

FIG. 2. The difference between A) systemic drug administration, B) systemic pulmonary drug administration and C) local pulmonary drug deposition

### DETAILED DESCRIPTION OF THE INVENTION

The present invention relates to the pulmonary administration, by any appropriate method including, but not limited to, intratracheal, intrabronchial, bronchio-alveolar or intraalveolar administration, e.g. via inhalation, to a human subject inclusive of both adults and children, of composition comprising an effective amount of one or more pro-regenerative factors, however prepared, to enhance airway epithelial repair and/or regeneration and/or the normalization of airway epithelial remodelling.

Administration of an effective amount of one or more regenerative factors via intratracheal, intrabronchial, intraalveolar or bronchio-alveolar administration is particularly useful in alleviating symptoms and/or treating subjects suffering from pulmonary conditions including, but not limited to Asthma, Chronic obstructive pulmonary disease (COPD), chronic bronchitis, Cystic fibrosis, Bronchiectasis, Panbronchiolitis, lung cancer, pneumonia, ventilator associated pneumonia (VAP), hospital acquired pneumonia (HAP), community acquired pneumonia (CAP), severe community acquired pneumonia (sCAP) or pneumocystis carinii.

### Epithelial Repair, Regeneration and Remodelling

As described herein above the airway epithelium may be rapidly disturbed after any infectious or inflammatory-related in chronic diseases such as asthma, chronic obstructive pulmonary disease (COPD), bronchietasis and cystic fibrosis.

The remodelling process following injury of the airway epithelium, include structural changes such as squamous cell and/or goblet cell hyperplasia/metaplasia (GCH/GCM), mucous hyperplasia, hypertrophy of submucosal glands, subepithelial fibrosis and hyperplasia and hypertrophy of airway smooth muscle cells. This leads to mucus hypersecretion and hypertrophied airway walls causing the characteristic limitation in airflow.

The non-ciliated serous Clara cells of the airway epithelium represent major progenitor cells for airway renewal in the small airways of humans. In chronic obstructive lung disease and or chronic bronchitis the airway lining is destroyed. This implies airway modeling with goblet cell transformation with highly viscous mucous and impaired cilial clearance of particles deposited in the airways and dead alveolar macrophages.

Without this cilial clearance mechanism the peripheral airways, i.e. bronchioles and alveoles are subjected to proteolytic degradation from the proteases released from dead alveolar macrophages. In contrast the clearance of mucous from the central airways only depends on the coughing of sputum, however, when the mucous has become highly viscous the central airway clearance is also impeded. So in all both the peripheral and the central clearance mechanisms are hampered following pathological airway remodeling after inflammatory insult to the airways as seen in COPD chronic lung disease and cystic fibrosis.

In chronic airways diseases like chronic obstructive pulmonary disease (COPD), chronic bronchitis, cystic fibrosis and asthma the above-mentioned airway remodeling may considerably disturb both the innate immune and the respiratory function of the airway epithelium.

In vitro (for example 2-D cell culture) and in vivo models (for example human airway xenograft in mice) of airway epithelial wound repair and regeneration allow the study of the so-called “spatiotemporal” modulation of cellular and molecular interaction factors, i.e. the proinflammatory cytokines, the matrix proteinases and their inhibitors, and the intercellular adhesion molecules. 2-D cell culture has for example shown that one of the first and most important events during airway re-epithelialization is cell migration and not proliferation. Human airway xenograft in mice has for example been used to study the role of cellular and molecular mechanisms of airway epithelial regeneration after injury.

Enhancement of the airway epithelial repair and/or regeneration and/or the normalization of airway epithelial remodeling can be determined, for example, by, measurements of clearance rate of insoluble radio-labeled markers deposited by the inhalation of aerosol delivery onto the mucus layer in the more distal airways. The clearance rate may subsequently be measured by scintigraphy monitoring local pulmonary airway epithelial repair and/or regeneration and/or the normalization of airway epithelial remodeling parameters such as changes in epithelial cell migration, cell proliferation, cell differentiation and cell function

In a preferred embodiment of the invention the administration one or more regenerative factors may alleviate or prevent any of the symptoms of Asthma, Chronic obstructive pulmonary disease (COPD), chronic bronchitis, Cystic fibrosis, Bronchiectasis, Panbronchiolitis, lung cancer, pneumonia, ventilator associated pneumonia (VAP), hospital acquired pneumonia (HAP), community acquired pneumonia (CAP), severe community acquired pneumonia (sCAP) or pneumocystis carinii as described herein below.

### Pro-Regenerative Factors

A pro-regenerative factor according to the present invention may be any factor that enhance epithelial repair with or without improved clearance function both in respect to central airways and most importantly from peripheral airways and alveoli, with airway restitution therapeutic potential could particularly include both a quantitative and qualitative change in sputum, i.e. reduction of the mucus production by suppression of transformation of cells into goblet cells with less viscid mucous properties. This in turn may improve mucus rheology with a less thick mucous production into a more serous production being less viscous. Further and such a therapy stimulates growth and regeneration of cilial cells. Taken together such an airway remodeling process will improve clearance mechanisms by reduced viscous mucous and improved ciliary clearance both improving patients symptoms and pulmonary function effectively.

A pro-regenerative factor according to the present invention includes but are not limited to defense proteins such as Defensin; surfactant proteins such as SP-A, SP-B, SP-D; mucins; sIgA, lactoferrin, glycosaminoglycans, Clara cell secretory protein (CCSP), tight junction proteins such as Z01, occluding, claudins and Hepatocyte growth factor (HGF) and trefoil factor family proteins.

Other pro-regenerative factors according to the present invention includes but are not limited to Cellular and molecular factors involved in airway repair and regeneration are modulated by Matrix metalloproteinases (MMPs), cytokines and growth factors released by epithelial and mesenchymal cells.

### Trefoil Family

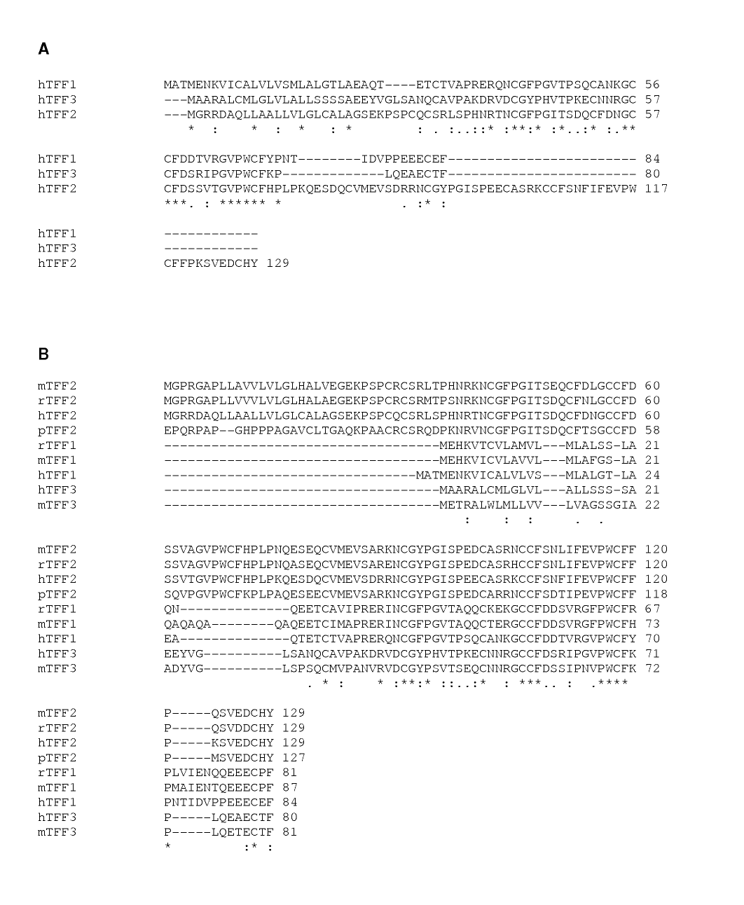

Reference herein to “TFF”, “TFF protein(s)”, “Trefoil family proteins” or “TFF family of proteins” refers to the group of related proteins including TFFI, TFF2, and TFF3. TFF proteins share at least approximately 28 to 45% amino acid identity within the same species (see FIGS. 1 A and B). All members of the trefoil family are comprised within the present invention including but not limited to human trefoil family factor 1 of SEQ ID NO.1, human trefoil family factor 2 of SEQ ID NO. 2 and human trefoil family factor 3 of SEQ ID NO.3.

The P-type trefoil or P-domain [KRH]-x(2)-C-x-[FYPSTV]-x(3,4)-[ST]-x(3)-C-x(4)-C-C-[FYWH] is a 45-residue cysteine-rich region, 6 cysteines of which link together through 3 disulphide bonds with connectivity 1-5, 2-4, 3-6, thus:

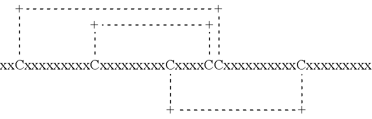

The domain is known as either the ‘P’, ‘trefoil’ or ‘TFF’ domain, and contains six cysteines linked by three disulphide bonds with connectivity 1-5, 2-4, 3-6. The domain has been found in a variety of extracellular eukaryotic proteins, including protein pS2 (TFF1), a protein secreted by the stomach mucosa; spasmolytic polypeptide (SP) (TFF2), a protein of about 115 residues that inhibits gastrointestinal motility and gastric acid secretion; intestinal trefoil factor (ITF) (TFF3).

In human TFF1 the TFF domain spans amino acid residues 30 through 70. Human TFF2 has two TFF domains spanning respectively amino acid residues 30 through 72 and amino acid residues 80 through 121. In human TFF3 the TFF domain spans amino acid residues 31 through 72.

Functional analysis of TFF3 has determined that its expression is sufficient to stimulate an increase in total cell number by concomitant increase in mitogenesis and cell survival, support anchorage independent growth of human carcinoma cells and other indices of oncogenicity including growth in suspension culture and foci formation. Furthermore, TFF supported oncogenic transformation of immortalized, but otherwise normal, human epithelial cells. siRNA mediated decrease of TFF3 expression concordantly abrogated anchorage independent growth of human carcinoma cells. These results indicate that inhibition of TFF expression and/or activity leads to a decrease in cellular proliferation and decreases the progression and severity of cancer and other proliferative disorders. Similar results can be shown with other members of the TFF family of proteins.

In preferred embodiments of the invention the pro-regenerative factor is a trefoil family protein including TFF1 of SEQ ID NO 1, TFF2 of SEQ ID No 2 and TFF3 of SEQ ID NO 3. In a very preferred embodiment the pro-regenerative factor is TFF3.

In other preferred embodiments of the invention the pro-regenerative factor is a dimer of TFF1 of SEQ ID NO 1, TFF2 of SEQ ID No 2 and TFF3 of SEQ ID NO 3. Dimers of TFF peptides consists of two TFF monomers linked together by a disulfide bond. For the TFF1 dimer, the disulfide bond may preferably be between cysteine amino acid at residue 58 of each TFF1 monomer. For the TFF3 dimer, the disulfide bond may preferably be between cysteine amino acid at residue 57 of each TFF3 monomer. TFF dimmers may for example be produced as described in WO 96/06861.

### Variants of TFF

A variant of a TFF peptide or a TFF dimer peptide is a polypeptide having at least 70% sequence identity with SEQ ID NO. 1 SEQ ID NO 2 or SEQ ID NO. 3 and has one or more TFF functions.

Preferably said variant of any of the predetermined sequences herein, may be functional variants such as SEQ ID NO: 1, SEQ ID NO 2 or SEQ ID NO: 3 defined as:
i) variants comprising an amino acid sequence capable of stimulating an increase in total cell number by concomitant increase in mitogenesis and cell survival
ii) variants capable of supporting anchorage independent growth of human carcinoma cells
iii) variants capable of growth in suspension culture and foci formation
iv) variants capable of enhancing airway epithelial repair and/or regeneration and/or normalization of airway epithelial remodeling

The ability of a variant or fragment of a TFF peptide to exhibit a biological activity of a TFF peptide or TFF dimer peptide can be assessed by methods known to those skilled in the art.

Methods for assaying the functional activity of TFF proteins for use in the present invention include those described in WO/2006/069253, which is hereby incorporated by reference.

TFF activity may also be determined by its ability to chemoattract human monocytes using a concentration range of 1.0-10.0 microgram/ml.

Preferably, evolutionary conservation between TFFs of different closely related species, e.g. assessed by sequence alignment, can be used to pinpoint the degree of evolutionary pressure on individual residues. Preferably, TFF sequences are compared between species where TFF function is conserved.

Residues under high selective pressure are more likely to represent essential amino acids that cannot easily be substituted than residues that change between species. It is evident from the above that a reasonable number of modifications or alterations of the human TFF sequence does not interfere with the activity of the TFF molecule according to the invention. Such TFF molecules are herein referred to as variants of human TFF, and may be such as variants and fragments of native human TFF as described herein.

As used herein the expression “variant” refers to polypeptides or proteins which are homologous to the basic protein, which is suitably human TFF1, TFF2 or TFF3, but which differs from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. FIG. 1B shows an alignment of the protein sequences of human TFF1, human TFF2, human TFF3, mouse TFF1, mouse TFF2, mouse TFF3, rat TFF1, rat TFF1 and pig TFF2 wherein identical residues (“*”) and residues with conservative (“:”) and semi-conservative (“.”) substitutions are marked.

Accordingly, in one embodiment of the invention it is preferred that a variant of human TFF1, TFF2 or TFF3, comprises a sequence with high sequence identity to SEQ ID NO: 1, SEQ ID NO: 2 or SEQ ID NO:3, wherein none of the conserved residues marked with “*” in FIG. 1B are substituted. It is furthermore preferred within this embodiment that the residues marked with “:” in FIG. 1B are either not substituted or only substituted by conservative substitution, more preferably by substitution with an amino acid with a high level of similarity as defined herein below.

Accordingly, in one embodiment of the invention it is preferred that a variant of human TFF1, TFF2 or TFF3, comprises a sequence with high sequence identity to SEQ ID NO: 1, SEQ ID NO: 2 or SEQ ID NO:3, wherein none of the conserved residues marked with “*” in FIG. 1A are substituted. It is furthermore preferred within this embodiment that the residues marked with “:” in FIG. 1A are either not substituted or only substituted by conservative substitution, more preferably by substitution with an amino acid with a high level of similarity as defined herein below.

Thus in one embodiment it is preferred that variants of human TFF1 have a sequence with high sequence identity to SEQ ID NO: 1, wherein residues M4, L12, L18, A24, R38, C41, G42, P44, T47, C51, G55, C56, C57, F58, D59, G64, V65, P66, W67, C68, F69, P71, C82, K7, V15, T22, E29, V33, R36, E37, N40, F43, V46, Q50, N53, V62, E81 and F84 are either not substituted or substituted only by conservative substitution, more preferably substituted only an amino acid with a high level of similarity as defined herein below.

In another embodiment it is preferred that variants of human TFF2 have a sequence with high sequence identity to SEQ ID NO: 2, wherein residues M1, L9, L15, A21, R39, C42, G43, P45, T48, C52, G56, D57, C58, F59, D60, G65, V66, P67, W68, C69, F70, P73, C92, R4, L12, A19, Q30, L34, H37, N38, N41, F44, I47, Q51, D54, V63, N91 and Y94 are either not substituted or substituted only by conservative substitution, more preferably substituted only an amino acid with a high level of similarity as defined herein below.

In yet another preferred embodiment it is preferred that variants of human TFF2 have a sequence with high sequence identity to SEQ ID NO: 2, wherein residues M1, L9, L15, A21, R39, C42, G43, P45, T48, C52, G56, D57, C58, F59, D60, G65, V66, P67, W68, C69, F70, P72, C79, F81, R4, V12, S19, N30, V34, K37, D38, D41, Y44, V47, E51, N54, I63, E77 are either not substituted or substituted only by conservative substitution, more preferably substituted only an amino acid with a high level of similarity as defined herein below.

It is even further preferred within the present invention that variants of TFF1, TFF2 or TFF3 have a sequence with high sequence identity to SEQ ID NO:1 or SEQ ID NO: 2, wherein residues marked with “.” in FIG. 1B are either not substituted or are only substituted by conservative substitutions, such as with amino acids with lower levels or high level of similarity as defined herein below.

It is even further preferred within the present invention that variants of TFF1, TFF2 or

TFF3 have a sequence with high sequence identity to SEQ ID NO:1 or SEQ ID NO: 2, wherein residues marked with “.” in FIG. 1A are either not substituted or are only substituted by conservative substitutions, such as with amino acids with lower levels or high level of similarity as defined herein below.

Accordingly, it is preferred that variants of human TFF1 have a sequence with high sequence identity to SEQ ID NO: 1, wherein A34, P35, P48, S49, K54, D60 and E79 are either not substituted or only substituted by conservative substitutions, such as with amino acids with lower level or high level of similarity as defined herein below. It is also preferred that variants of human TFF2 have a sequence with high sequence identity to SEQ ID NO: 2, wherein S32, S35, P36, S49, D50, N55, S61 and R89 are either not substituted or only substituted by conservative substitutions, such as with amino acids with lower level or high level of similarity as defined herein below. It is furthermore preferred that variants of human TFF3 have a sequence with high sequence identity to SEQ ID NO: 3, wherein C32, P35, A36, P49, K50, R55, S61 and E75 are either not substituted or only substituted by conservative substitutions, such as with amino acids with lower level or high level of similarity as defined herein below.

It is also comprised within the present invention that variants of TFF1, TFF2 or TFF3 may have a sequence with high sequence identity to SEQ ID NO: 1, SEQ ID NO: 2 or SEQ ID NO:3, wherein the unmarked residues in FIG. 1B may be substituted with any other amino acid.

It is also comprised within the present invention that variants of TFF1, TFF2 or TFF3 may have a sequence with high sequence identity to SEQ ID NO: 1, SEQ ID NO: 2 or SEQ ID NO:3, wherein the unmarked residues in FIG. 1A may be substituted with any other amino acid.

Thus, variants of human TFF1 may have a sequence with high sequence identity to SEQ ID NO: 1, wherein residues M1, A2, T3, E5, N6, V8, I9, C10, A11, V13, L14, Q39, G45, A52, T61, R63, Y70, N72, T73, I74, D75, V76, P77, P78, E80 and E83 are either not substituted or substituted with any other amino acid. Thus, variants of human TFF2 may have a sequence with high sequence identity to SEQ ID NO: 2, wherein residues G2, R3, D5, A6, Q7, L8, A10, A11, L13, V14, G16, L17, C18, L20, G21, S23, E24, K25, P26, S27, P28, C29, C31, R33, T40, G46, F53, S62, T64, H71, L73, P74, K75, Q76, E77, S78, D79, Q80, C81, V82, M83, E84, V85, S86, D87, R89, G92, P94, G95, I96, S97, P98, E99, E100, C101, A102, S103, R104, K105, C106, C107, F108, S109, N110, F111, I112, F113, E114, V115, P116 and W117 are either not substituted or substituted with any other amino acid. Thus, variants of human TFF3 may have a sequence with high sequence identity to SEQ ID NO: 3, wherein residues A2, A3, A5, L6, C7, M8, G10, L11, L13, A14, L16, S17, S18, S20, E22, E23, Y24, V25, G26, L27, S28, A29, Q31, A33, V40, H46, N53, R62, P64, K71, L73, Q74, A76 and T79 are either not substituted or substituted with any other amino acid.

In other preferred embodiments of the invention the TFF domain of TFF1, TFF2 or TFF3 of SEQ ID NO: 1, SEQ ID NO: 2 or SEQ ID NO:3, are either not substituted or substituted only by conservative substitution, more preferably substituted only an amino acid with a high level of similarity as defined herein below. Thus in these embodiments variants of human TFF1 have a sequence with high sequence identity to SEQ ID NO: 1, wherein amino acids 30 through 70 are either not substituted or only substituted by conservative substitutions, such as with amino acids with lower level or high level of similarity as defined herein below; variants of human TFF2 have a sequence with high sequence identity to SEQ ID NO: 2, wherein amino acids 30 through 72 and amino acids 80 through 121 are either not substituted or only substituted by conservative substitutions, such as with amino acids with lower level or high level of similarity as defined herein below; variants of human TFF3 have a sequence with high sequence identity to SEQ ID NO: 3, wherein amino acids 31 through 72 are either not substituted or only substituted by conservative substitutions, such as with amino acids with lower level or high level of similarity as defined herein below.

A person skilled in the art will know how to make and assess ‘conservative’ amino acid substitutions, by which one amino acid is substituted for another with one or more shared chemical and/or physical characteristics. Conservative amino acid substitutions are less likely to affect the functionality of the protein. Amino acids may be grouped according to shared characteristics. A conservative amino acid substitution is a substitution of one amino acid within a predetermined group of amino acids for another amino acid within the same group, wherein the amino acids within a predetermined groups exhibit similar or substantially similar characteristics. Within the meaning of the term “conservative amino acid substitution” as applied herein, one amino acid may be substituted for another within groups of amino acids characterized by having similar chemophysical characteristics (i.e. polarity, hydrophilicity, hydrophobicity, charges or other features as listed below).

Within the meaning of the term “conservative amino acid substitution” as applied herein, one amino acid may be substituted for another within the groups of amino acids indicated herein below:

### Lower Levels of Similarity:

### Polarity:

i) Amino acids having polar side chains (Asp, Glu, Lys, Arg, His, Asn, Gln, Ser, Thr, Tyr, and Cys,)
ii) Amino acids having non-polar side chains (Gly, Ala, Val, Leu, Ile, Phe, Trp, Pro, and Met)

### Hydrophilic or Hydrophobic:

iii) Hydrophobic amino acids (Ala, Cys, Gly, Ile, Leu, Met, Phe, Pro, Trp, Tyr, Val)
iv) Hydrophilic amino acids (Arg, Ser, Thr, Asn, Asp, Gln, Glu, His, Lys)

### Charges:

v) Neutral amino acids (Ala, Asn, Cys, Gln, Gly, Ile, Leu, Met, Phe, Pro, Ser, Thr, Trp, Tyr, Val)
vi) Basic amino acids (Arg, His, Lys)
vii) Acidic amino acids ((asp, Glu)

### High Level of Similarity:

viii) Acidic amino acids and their amides (Gln, Asn, Glu, Asp)
ix) Amino acids having aliphatic side chains (Gly, Ala Val, Leu, Ile)
x) Amino acids having aromatic side chains (Phe, Tyr, Trp)
xi) Amino acids having basic side chains (Lys, Arg, His)
xii) Amino acids having hydroxy side chains (Ser, Thr)
xiii) Amino acids having sulphur-containing side chains (Cys, Met),
   Preferred conservative amino acids substitution groups are: valine-leucine-isoleucine, phenylalanine-tyrosine, lysine-arginine, alanine-valine, and asparagine-glutamine.

A variant within the scope of the present invention is a polypeptide that exhibits at least 70% sequence identity with human TFF1, TFF2 or TFF3 as identified by respectively SEQ ID NO. 1, SEQ ID NO. 2 or SEQ ID NO. 3, preferably variants have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with SEQ ID NO. 1, SEQ ID NO. 2 or SEQ ID NO. 3.

Sequence identity can be calculated using a number of well-known algorithms and applying a number of different gap penalties. Any sequence alignment algorithm, such as but not limited to FASTA, BLAST, or GETSEQ may be used for searching homologues and calculating sequence identity. Moreover, when appropriate any commonly known substitution matrix, such as but not limited to PAM, BLOSSUM or PSSM matrices, may be applied with the search algorithm. For example, a PSSM (position specific scoring matrix) may be applied via the PSI-BLAST program. Moreover, sequence alignments may be performed using a range of penalties for gap opening and extension. For example, the BLAST algorithm may be used with a gap opening penalty in the range 5-12, and a gap extension penalty in the range 1-2.

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

Aside from the twenty standard amino acids and two special amino acids, selenocysteine and pyrrolysine, there are a vast number of “nonstandard amino acids” which are not incorporated into protein in vivo. Examples of nonstandard amino acids include the sulfur-containing taurine and the neurotransmitters GABA and dopamine. Other examples are lanthionine, 2-Aminoisobutyric acid, and dehydroalanine. Further non standard amino are ornithine and citrulline.

Non-standard amino acids are usually formed through modifications to standard amino acids. For example, taurine can be formed by the decarboxylation of cysteine, while dopamine is synthesized from tyrosine and hydroxyproline is made by a posttranslational modification of proline (common in collagen). Examples of non-natural amino acids are those listed e.g. in 37 C.F.R. section 1.822(b) (4), all of which are incorporated herein by reference.

Both standard and non standard amino acid residues described herein can be in the “D” or or “L” isomeric form.

It is contemplated that a variant according to the invention may comprise any amino acid including non-standard amino acids. In preferred embodiments a variant comprises only standard amino acids.

The standard and/or non-standard amino acids may be linked by peptide bonds or by non-peptide bonds. The term peptide also embraces post-translational modifications introduced by chemical or enzyme-catalyzed reactions, as are known in the art. Such post-translational modifications can be introduced prior to partitioning, if desired. Amino acids as specified herein will preferentially be in the L-stereoisomeric form. Amino acid analogs can be employed instead of the 20 naturally-occurring amino acids. Several such analogs are known, including fluorophenylalanine, norleucine, azetidine-2-carboxylic acid, S-aminoethyl cysteine, 4-methyl tryptophan and the like.

Variants may further comprise chemical modifications such as ubiquitination, labeling (e.g., with radionuclides, various enzymes, etc.), pegylation (derivatization with polyethylene glycol), or by insertion (or substitution by chemical synthesis) of amino acids (amino acids) such as ornithine, which do not normally occur in human proteins.

In addition to the peptidyl compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Variants also comprise glycosylated and covalent or aggregative conjugates formed with the same molecules, including dimers or unrelated chemical moieties. Such variants are prepared by linkage of functionalities to groups which are found in fragment including at any one or both of the N- and C-termini, by means known in the art.

The term “fragment thereof” may refer to any portion of the given amino acid sequence and variants thereof. Fragments may comprise more than one portion from within the full-length protein, joined together. Portions will suitably comprise at least 5 and preferably at least 10 consecutive amino acids from the basic sequence. They may include small regions from the protein or combinations of these

Suitable fragments may be addition mutants. The addition of at least one amino acid may be an addition of from preferably 2 to 250 amino acids, such as from 10 to 20 amino acids, for example from 20 to 30 amino acids, such as from 40 to 50 amino acids. Fragments may include small regions from the protein or combinations of these.

Suitable fragments may be deletion mutants capable of enhancing airway epithelial repair and/or regeneration and/or the normalization of airway epithelial remodeling. The deletion of at least one amino acid may be a deletion of from preferably 2 to 180 amino acids, such as from 10 to 20 amino acids, for example from 20 to 30 amino acids, such as from 40 to 50 amino acids. The deletion and/or the addition may—independently of one another—be a deletion and/or an addition within a sequence and/or at the end of a sequence.

Deletion mutants suitably comprise at least 20 or 40 consecutive amino acid and more preferably at least 80 or 100 consecutive amino acids in length. Accordingly such a fragment may be a shorter sequence of the sequence as identified by SEQ ID NO. 1, SEQ ID NO. 2 or SEQ ID NO. 3 comprising at least 20 consecutive amino acids, for example at least 30 consecutive amino acids, such as at least 40 consecutive amino acids, for example at least 50 consecutive amino acids, such as at least 60 consecutive amino acids, for example at least 70 consecutive amino acids, such as at least 80 consecutive amino acids, for example at least 90 consecutive amino acids, such as at least 95 consecutive amino acids, such as at least 100 consecutive amino acids, such as at least 105 amino acids, for example at least 110 consecutive amino acids, such as at least 115 consecutive amino acids, for example at least 120 consecutive amino acids, wherein said deletion mutants preferably has at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with SEQ ID NO. 1, SEQ ID NO. 2 or SEQ ID NO. 3.

Preferably, the number of substitutions, deletions, or additions is 20 amino acids or less, such as 15 amino acids or less, for example 10 amino acids or less, such as 9 amino acids or less, for example 8 amino acids or less, such as 7 amino acids or less, for example 6 amino acids or less, such as 5 amino acids or less, for example 4 amino acids or less, such as 3 amino acids or less, for example 2 amino acids or less (such as 1), or any integer in between these amounts. In one aspect of the invention, the substitutions include one or more conservative substitutions, such as 20 or fewer conservative substitutions, for example 18 or fewer, such as 16 or fewer, for example 14 or fewer, such as 12 or fewer, for example 10 or fewer, such as 8 or fewer, for example 6 or fewer, such as 4 or fewer, for example 3 or fewer, such as 2 or fewer conservative substitutions.

Variants according to the present invention may be insertional variants in which one or more amino acid residues are introduced into a predetermined site in TFF. For instance, insertional variants can be fusions of heterologous proteins or polypeptides to the amino or carboxyl terminus of the subunits.

### Medical Indications

Administration of an effective amount of one or more pro-regenerative factors, preferably a TFF member, a TFF dimer, more preferably TFF3 or TFF 3 dimer via intratracheal, intrabronchial, intraalveolar or bronchio-alveolar administration is particularly useful in enhancing epithelial repair and thereby alleviating symptoms and/or be useful for treating subjects suffering from conditions including, but not limited to Asthma, Chronic obstructive pulmonary disease (COPD), chronic bronchitis, Cystic fibrosis, Bronchiectasis, Panbronchiolitis, lung cancer, pneumonia, ventilator associated pneumonia (VAP), hospital acquired pneumonia (HAP), community acquired pneumonia (CAP), severe community acquired pneumonia (sCAP) or pneumocystis carinii.

The patients may display symptoms such as fever, infiltrates on chest x-ray, dyspnea, coughing or purulent sputum, increased level of biomarkers, e.g. erythrocyte sedimentation rate (ESR), procalcitonin test (PCT) and C-reactive protein (CRP) and pulmonary dysfunction even with reduced oxygenation capacity as revealed by a reduced PaO₂/FiO₂ ratio (arterial oxygen tension in mmHg over inspired oxygen fraction) concomitant with or in spite of treatment with full antibiotic coverage towards the isolated microbiological agent or treatment of underlying disease. In a preferred embodiment of the invention the administration of one or more pro-regenerative factors may alleviate or prevent any of the above symptoms of Asthma, Chronic obstructive pulmonary disease (COPD), chronic bronchitis, Cystic fibrosis, Bronchiectasis, Panbronchiolitis, lung cancer, pneumonia, ventilator associated pneumonia (VAP), hospital acquired pneumonia (HAP), community acquired pneumonia (CAP), severe community acquired pneumonia (sCAP) or pneumocystis carinii.

In one embodiment the patient may experience acute exacerbations in for example chronic obstructive pulmonary disease (COPD) caused by infection. Administration of an effective amount of one or more pro-regenerative factors via intratracheal, intrabronchial, intraalveolar or bronchio-alveolar administration is also useful in alleviating symptoms and/or treating acute exacerbations in for example chronic obstructive pulmonary disease (COPD) caused by infection, such as bacterial infection.

Infections may for example be an infection by bacteria, fungi, viruses, parasites. For example infection by parasites such as plasmodium falciparum. For example infection by one or more bacteria selected from the group consisting of Achromobacter xylosoxidans, Acinetobacter calcoaceticus, preferably A. anitratus, A. haemolyticus, A. alcaligenes, and A. lwoffii, Actinomyces israelii, Aeromonas hydrophilia, Alcaligenes species, preferably A. faecalis, A. odorans and A. denitrificans, Arizona hinshawii, Bacillus anthracis, Bacillus cereus, Bacteroides fragilis, Bacteroides melaminogenicus, Bordetella pertussis, Borrelia burgdorferi, Borrelia recurrentis, Brucella species, preferably B. abortus, B. suis, B. melitensis and B. canis, Calymmatobacterium granulomatis, Campylobacter fetus ssp. intestinalis, Campylobacter fetus ssp. jejuni, Chlamydia species, preferably C. psittaci and C. trachomatis, Chromobacterium violaceum, Citrobacter species, preferably C. freundii and C. diversus, Clostridium botulinum, Clostridium perfringens, Clostridium difficile, Clostridium tetani, Corynebacterium diphtheriae, Corynebacterium, preferably C. ulcerans, C. haemolyticum and C. pseudotuberculosis, Coxiella burnetii, Edwardsiella tarda, Eikenella corrodens, Enterobacter, preferably E. cloacae, E. aerogenes, E. hafniae (also named Hafnia alvei) and E. agglomerans, Erysipelothrix rhusiopathiae, Escherichia coli, Flavobacterium meningosepticum, Francisella tularensis, Fusobacterium nucleatum, Gardnerella vaginalis, Haemophilus ducreyi, Haemophilus influenzae, Helicobacter species, Klebsiella species, preferably K. pneumoniae, K. ozaenae og K. rhinoscleromatis, Legionella species, Leptospira interrogans, Listeria monocytogenes, Moraxella species, preferably M. lacunata and M. osloensis, Mycobacterioum bovis, Mycobacterium leprae, Mycobacterium tuberculosis, Mycoplasma species, preferably M. pneumoniae, Neisseria gonorrhoeae, Neisseria meningitidis, Nocardia species, preferably N. asteroides and N. brasiliensis, Pasterurella haemolytica, Pasteurella multocida, Peptococcus magnus, Plesiomonas shigelloides, Pneumococci, Proteus species, preferably P. mirabilis, P. vulgaris, P. rettgeri and P. morganii (also named Providencia rettgeri and Morganella morganii respectively), Providencia species, preferably P. alcalifaciens, P. stuartii and P. rettgeri (also named Proteus rettgeri), Pseudomonas aeruginosa, Pseudomonas mallei, Pseudomonas pseudomallei, Rickettsia, Rochalimaia henselae, Salmonella species, preferably S. enteridis, S. typhi and S. derby, and most preferably Salmonella species of the type Salmonella DT104, Serratia species, preferably S. marcescens, Shigella dysenteriae, S. flexneri, S. boydii and S. sonnei, Spirillum minor, Staphylococcus aureus, Staphylococcus epidermidis, Staphylococcus saprophyticus, Streptobacillus moniliformis, Streptococcus, preferably S. faecalis, S. faecium and S. durans, Streptococcus agalactiae, Streptococcus pneumoniae, Streptococcus pyogenes, Treponema carateum, Treponeam pallidum, Treponema pertenue, preferably T. pallidum, Ureaplasma urealyticum, Vibrio cholerae, Vibrio parahaemolyticus, Yersinia enterocolitica, and Yersinia pestis.

Infections also comprise protozoan infections such as, but not limited to, Trichomonas infections, such as Pentatrichomonas infections. For example T. buccalis, T. tenax, T. foetus, T. gallinae, T. gallinarum, T. hominis, T. intestinalis, T. tenax, T. vaginalis.

In other embodiments of the invention pro-regenerative factors may be used for the treatment of any condition caused by fungal infections including, but not limited to: Aspergillosis, Blastomycosis, Candidiasis, Coccidioidomycosis, Cryptococcosis, Histoplasmosis, Paracoccidiomycosis, Sporotrichosis, Zygomycosis. The composition may also be used to treat fungal infections in conditions such as Chromoblastomycosis, Mycotic keratitis, Endogenous oculomycosis, Extension oculomycosis, Lobomycosis, Mycetoma, Nail, Hair, and Skin diseases (for example Onychomycosis (Tinea unguium), Piedra, Pityriasis versicolor, Tinea barbae, Tinea capitis, Tinea corporis, Tinea cruris, Tinea favosa, Tinea nigra, Tinea pedis), Otomycosis, Phaeohyphomycosis, Rhinosporidiosis.

In preferred embodiments the subject is suffering from a disease selected from the group consisting of Asthma, Chronic obstructive pulmonary disease (COPD), chronic bronchitis, Cystic fibrosis, Bronchiectasis, Panbronchiolitis, lung cancer, pneumonia, ventilator associated pneumonia (VAP), hospital acquired pneumonia (HAP), community acquired pneumonia (CAP), severe community acquired pneumonia (sCAP) or pneumocystis carinii.

### Pulmonary Administration

When used herein the term “deposition of drugs in the lungs” and other variations on this theme is meant to describe local pulmonary drug administration and not systemic drug administration via the airways with the purpose of ensuring a true local effect by eliminating or at least minimizing the systemic spill over of the pro-regenerative factor, preferably a TFF member and/or a TFF dimer, more preferably TFF3 in order to avoid or minimize the many adverse effects connected with systemic administration of pro-regenerative factor, preferably a TFF member, and/or a TFF dimer, more preferably TFF3 or TFF 3 dimer.

To optimize deposition on the ciliated mucosa in bronchi and the peripheral airways (small airways) using e.g. inhalation, a key determining variable is the aerosol particle size. It is generally accepted that for particles with aerodynamic diameters of 1-4 microns a distal deposition is also ensured. This factor is highly important, because the transition from the alveoli to bronchiole is solely dependent on proper cilia function. Accumulation of dead cells or cell debris with otherwise liberates proteolytic enzymes into the alveolar space giving rise to severe inflammation fibrosis and emphysema. Larger aerosol diameter ranges like ˜>5 micron will, however, not reach this crucial part of the airways and will only be deposited in the more central airways.

For inhaled formulations, the balance between desired local effects and undesired systemic activity can be expressed by L/T, where L represents bioavailability of drug from the lungs and T represents total systemic bioavailability. A high L/T. e.g. obtained by inhalation, is desirable as this implies efficient amounts of drug delivery to the target site, where the clearing effect of coughing i.e. is only dependent on the cilial function and also minimization of unwanted activity from non-targeted drug delivery.

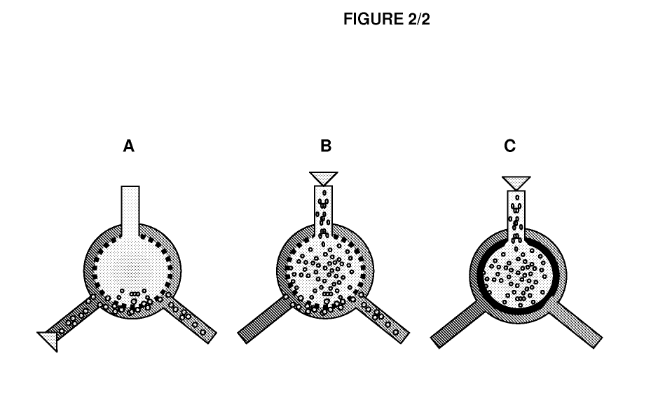

A schematic representation of the difference between systemic drug administration, systemic pulmonary drug administration and local pulmonary drug deposition can be seen in FIG. 2.

Methods of intratracheal, intrabronchial, intraalveolar or bronchio-alveolar administration include, but are not limited to, inhalation of aerosolized solution, dry powder inhalation, spraying, lavage, flushing or instillation, using as fluid a physiologically acceptable composition in which one or more pro-regenerative factors have been dissolved. When used herein the terms “inhalation, intratracheal, intrabronchial or intraalveolar or bronchio-alveolar administration” include all forms of such local pulmonary administration whereby one or more pro-regenerative factors is applied into the trachea, the bronchi or the alveoli, respectively, whether by the instillation of a solution of one or more pro-regenerative factors, by applying one or more pro-regenerative factors in a powder form, or by allowing one or more pro-regenerative factors to reach the relevant part of the airway by inhalation of one or more pro-regenerative factors as an aerosolized or nebulized solution or suspension or inhaled powder or gel, with or without added stabilizers or other excipients.

Methods of intrabronchial/alveolar administration include, but are not limited to, bronchoalveolar lavage (BAL) according to methods well known to those skilled in the art, using as a lavage fluid a physiologically acceptable composition in which one or more pro-regenerative factors been dissolved or indeed by any other effective form of intrabronchial administration including the use of inhaled powders containing one or more pro-regenerative preferably a TFF member, and/or a TFF dimer, more preferably TFF3 or TFF 3 dimer in dry form, with or without excipients, or the direct application of one or more pro-regenerative factors preferably a TFF member, and/or a TFF dimer, more preferably TFF3 or TFF 3 dimer, in solution or suspension or powder form during bronchoscopy. Methods for intratracheal administration include, but are not limited to, blind tracheal washing with a similar solution of one or more dissolved pro-regenerative factors preferably a TFF member, and/or a TFF dimer, more preferably TFF3 or TFF 3 dimer or a suspension of one or more pro-regenerative factors preferably a TFF member, and/or a TFF dimer, more preferably TFF or TFF 3 dimer, or the inhalation of nebulized fluid droplets containing one or more dissolved pro-regenerative factors preferably a TFF member, and/or a TFF dimer, more preferably TFF3 or TFF 3 dimer, a suspension of one or more pro-regenerative factors, obtained by use of an nebulizing apparatus adequate for this purpose. The quality of such a nebulizer is characterized as a nebulizer which does not modify or interfere with the active sites of the inhaled molecule, e.g. by applying an electronic, mechanically or compressed air driven micropump nebulizer.

In another embodiment, intratracheal, intrabronchial or intraalveolar administration does not include inhalation of one or more pro-regenerative factors preferably a TFF member, and/or a TFF dimer, more preferably TFF3 or TFF 3 dimer but the inhalation or instillation or application of a solution of one or more pro-regenerative factors preferably a TFF member, and/or a TFF dimer, more preferably TFF3 or TFF 3 dimer or a powder or a gel containing one or more pro-regenerative factors into the trachea or lower airways.

Other preferred methods of administration may include using the following devices:
1. Electronic, mechanically or compressed air driven micropump nebulizers (e.g. The Pari Aeroneb Professional Nebulizer, Pari nebulizer)
2. Ultrasonic nebulizers
3. Pressurized nebulizers using compressed air/oxygen mixture
4. Metered dose inhaler (MDI)
5. Dry powder inhaler systems (DPI),
6. Unit dose inhalant

The aerosol may be delivered by via a) a mouthpiece, b) a facemask or c) via endotracheal tubes in intubated patients during mechanical ventilation (device 1, 2 and 3). The devices 1, 4, 5 and 6 may also be used by the self administration by the patient without assistance.

Thus, in one embodiment the effective amount of one or more pro-regenerative factors preferably a TFF member, and/or a TFF dimer, more preferably TFF3 or TFF 3 dimer is administered by preferably inhalation, but intratracheal, intrabronchial, intraalveolar or bronchio-alveolar administration may be applied.

In another embodiment the subject is administered a solution of one or more pro-regenerative factors preferably a TFF member, and/or a TFF dimer, more preferably TFF3 or TFF 3 dimer via bronchoalveolar lavage.

In another embodiment the subject is administered a solution of one or more pro-regenerative factors preferably a TFF member, and/or a TFF dimer, more preferably TFF3 or TFF 3 dimer via blind tracheal washing.

In another embodiment the subject is administered a nebulized solution or a suspension of one or more pro-regenerative factors preferably a TFF member, more preferably TFF3 or TFF 3 dimer.

In another embodiment the subject is administered a nebulized aerosol or inhaled powder form of one or more pro-regenerative factors preferably a TFF member, and/or a TFF dimer, more preferably TFF3 or TFF 3 dimer.

In another embodiment the subject is administered a pegylated, liposomal or nanoparticle prepared form of one or more pro-regenerative factors preferably a TFF member, more preferably TFF3.

In another embodiment the subject is administered one or more pro-regenerative factors preferably a TFF member, and/or a TFF dimer, more preferably TFF3 or TFF 3 dimer by direct application during bronchoscopy.

In another embodiment the subject is a mammal.

In another embodiment the mammal is a human.

In another embodiment the human is a child younger than 12 years of age.

In another embodiment the human is an adult older than 12 years of age.

Preferred concentrations for a solution comprising one or more pro-regenerative factors and/or variants of pro-regenerative factors preferably a TFF member, and/or a TFF dimer, more preferably TFF3 or TFF 3 dimer may vary widely i.e. from about 5% to about 100% by weight. A typical concentration is in the range from about 50% to about 100% by weight.

Preferred concentrations may be in the range of 0.1 μg to 10000 μg active ingredient per ml solution. The suitable concentrations are often in the range of from 0.1 μg to 5000 μg per ml solution, such as in the range of from about 0.1 μg to 3000 μg per ml solution, and especially in the range of from about 0.1 μg to 1000 μg per ml solution, such as in the range of from about 0.1 μg to 250 μg per ml solution. A preferred concentration would be from about 0.1 to about 5.0 mg, preferably from about 0.3 mg to about 3.0 mg, such as from about 0.5 to about 1.5 mg and especially in the range from 0.8 to 1.0 mg per ml solution. A preferred concentration would be from about 0.1 to about 5.0 mg, preferably from about 0.3 mg to about 3.0 mg, such as from about 0.2 to about 2.5 mg and especially in the range from 0.2 to 1.0 mg per ml solution.

### Pharmaceutical Composition

Pharmaceutical compositions or formulations for use in the present invention include one or more pro-regenerative factor, preferably a TFF member, and/or a TFF dimer more preferably TFF3 or TFF 3 dimer alone or in combination as a dry powder preparation, however, preferably dissolved in a pharmaceutically acceptable carrier, preferably an aqueous carrier or diluent, or as a pegylated preparation carried to the lower airways or as a liposomal or nanoparticle preparation administered as an aerosol via inhalation, or as a lavage fluid administered via a bronchoscope as a bronchoalveloar lavage or as a blind intratracheal wash or lavage. A variety of aqueous carriers may be used, including, but not limited to 0.9% saline, buffered saline, physiologically compatible buffers and the like. The compositions may be sterilized by conventional techniques well known to those skilled in the art. The resulting aqueous solutions may be packaged for use or filtered under aseptic conditions and freeze-dried, the freeze-dried preparation being dissolved in a sterile aqueous solution prior to administration

In one embodiment a freeze-dried preparation of one or more pro-regenerative factor, preferably a TFF member, more preferably TFF3 or TFF 3 dimer may be pre-packaged for example in single dose units. In an even more preferred embodiment the single dose unit is adjusted to the patient.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

The formulations may contain pharmaceutically acceptable carriers and excipients including microspheres, liposomes, microcapsules, nanoparticles or the like. Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipids or modifiers of liposomes are preferred e.g. in combination with cholesterol and/or phosphatidylcholine.

Long-circulating liposomes are characterized by their ability to extravasate at body sites where the permeability of the vascular wall is increased. The most popular way of producing long-circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipids applicable for liposomes are supplied by Avanti, Polar Lipids, Inc, Alabaster, Ala. Additionally, the liposome suspension may include lipid-protective agents which protect lipids against free-radical and lipid-peroxidative damage on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution.

Common surfactants well known to one of skill in the art can be used in the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

In some embodiments the micellar formulation may be mixed with propellants such as tetrafluoroethane, heptafluoroethane, dimethylfluoropropane, tetrafluoropropane, butane, isobutane, dimethyl ether and other non-CFC and CFC propellants, especially when delivered (e.g. applied to the buccal mucosa) through aerosol devices, e.g. metered dose inhalers (MDIs).

In some cases, it will be advantageous to include a compound, which promotes delivery of the active substance to its target.

In some embodiments the compositions of the present invention are formulated as aerosols. In order to efficiently reach the lung, the formulation maybe atomized into particles having aerodynamic sizes between approximately 1 and 10, preferably between 2 and 5 micrometers Such aerosols may generally comprise one or more agents capable of inhibiting angiogenesis, one or more propellants and either a surfactant or a solvent.

Thus in certain embodiments the compositions according to the present invention may comprise a propellant including but not limited to fluorocarbons and hydrogen-containing chlorofluorocarbons, and a number of medicinal aerosol formulations using such propellant systems are disclosed in, for example, EP 0372777, WO91/04011, WO91/11173, WO91/11495 and WO91/14422.

Suitable solvents for the pharmaceutical preparation within the scope of the present inventions are solutions containing at least 70% (v/v) of ethanol; solutions containing at least 85% (v/v) are preferred whilst solutions having an ethanol content of more than 95% (v/v) are particularly preferred. The concentration is given in percent by volume (v/v), the remainder being water. Most particularly preferred is ethanol which already contains small amounts of water, e.g. 96% ethanol, so that it is no longer hygroscopic and evaporates azeotropically.

One alternative is the development of nebulizers in which aqueous solutions of pharmacologically-active substances are sprayed under high pressure so as to produce a mist of inhalable particles. The advantage of these nebulizers is that there is no need to use any propellant gases whatsoever. Solutions of defined volumes containing active substances are sprayed, using high pressures through small nozzles to produce inhalable aerosols with a preferred particle size of between 1 and 10, preferably between 2 and 5 micrometers.

### Dose

According to the present invention a pharmaceutically effective amount or a therapeutically effective amount is to be understood as an amount sufficient to induce a desired biological result. The result can be enhancement of epithelial repair or regeneration and/or the normalization of airway epithelial remodeling and/or alleviation of the signs, symptoms, or causes of a disease, for example of Asthma, Chronic obstructive pulmonary disease (COPD), chronic bronchitis, Cystic fibrosis, Bronchiectasis, Panbronchiolitis, lung cancer, pneumonia, ventilator associated pneumonia (VAP), hospital acquired pneumonia (HAP), community acquired pneumonia (CAP), severe community acquired pneumonia (sCAP) or pneumocystis carinii, for example, an effective amount is generally that which provides either subjective relief of symptoms or an objectively identifiable improvement as noted by the clinician or other qualified observer, preferably such a relief of symptoms is a significant relief. Thus, by “effective amount” one or more pro-regenerative factors preferably a TFF member, more preferably TFF3, it is meant a dose, which, when administered via pulmonary administration, achieves a concentration of one or more pro-regenerative factors preferably a TFF member, more preferably TFF3 in the subject's airways and/or lung parenchyma which airway epithelial repair and/or regeneration and/or the normalization of airway epithelial remodeling.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg (microgram) active ingredient per administration with a preferred range of from about 0.1 μg to 10000 μg per kilo body weight.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 10000 μg per kilo body weight. Using monomeric forms of the compounds, the suitable dosages are often in the range of from 0.1 μg to 5000 μg per kilo body weight, such as in the range of from about 0.1 μg to 3000 μg per kilo body weight, and especially in the range of from about 0.1 μg to 1000 μg per kilo body weight. Using multimeric forms of the compounds, the suitable dosages are often in the range of from 0.1 μg to 1000 μg per kilo body weight, such as in the range of from about 0.1 μg to 750 μg per kilo body weight, and especially in the range of from about 0.1 μg to 500 μg per kilo body weight such as in the range of from about 0.1 μg to 250 μg per kilo body weight.

Administration may be performed once or may be followed by subsequent administrations. 0.1 μg to 5000 μg per kilo body weight, such as in the range of from about 0.1 μg to 3000 μg per kilo body weight, and especially in the range of from about 0.1 μg to 1000 μg per kilo body weight, preferably in the range of 5 μg to 500 μg, even more about 50 μg to about 200 μg administered via inhalation once or twice daily. The dosage will vary with the age, sex and weight of the subject to be treated. A preferred dosage of multimeric forms would be in the interval 1 mg to 70 mg per 70 kg body weight.

Suitable daily dosage ranges are per kilo body weight per day normally of the order of several hundred μg active ingredient per day with a preferred range of from about 0.1 μg to 10000 μg per kilo body weight per day. Using monomeric forms of the compounds, the suitable dosages are often in the range of from 0.1 μg to 5000 μg per kilo body weight per day, such as in the range of from about 0.1 μg to 3000 μg per kilo body weight per day, and especially in the range of from about 0.1 μg to 1000 μg per kilo body weight per day, when based on monomeric forms having a sequence identical to SEQ ID NO. 1, SEQ ID NO. 2 or SEQ ID NO. 3, for variants and fragments the dose is calculated based on the molecular weight of the monomeric form to the molecular weight of the homologues or fragments.

A unit dosage of the composition may contain from about 1 mg to about 200 mg, typically from about 25 mg to about 75 mg, such as about 50 mg of the pro-regenerative factor, preferably a TFF member and/or a TFF dimer, most preferably TFF 3 or TFF 3 dimer.

Duration of dosing will typically range from 1 day to about 4 months, such as 2 days to about 3 months, for example in the range of 1-2 days to 2 months, such as in the range of 1-2 days to 1 month.

### Medical Packaging

The compounds used in the invention may be administered alone or in combination with pharmaceutically acceptable carriers or excipients, in either single or multiple doses. The formulations may conveniently be presented in unit dosage form by methods known to those skilled in the art.

It is preferred that the compounds according to the invention are provided in a kit. Such a kit typically contains an active compound in dosage forms for administration. A dosage form contains a sufficient amount of active compound such that a desirable effect can be obtained when administered to a subject.

Thus, it is preferred that the medical packaging comprises an amount of dosage units corresponding to the relevant dosage regimen. Accordingly, in one embodiment, the medical packaging comprises a pharmaceutical composition comprising a compound as defined above or a pharmaceutically acceptable salt thereof and pharmaceutically acceptable carriers, vehicles and/or excipients, said packaging comprising from 1 to 7 dosage units, thereby having dosage units for one or more days, or from 7 to 21 dosage units, or multiples thereof, thereby having dosage units for one week of administration or several weeks of administration.

The dosage units can be as defined above. The medical packaging may be in any suitable form for intratracheal, intrabronchial, bronchio-alveolar or intraalveolar administration. In a preferred embodiment the packaging is in the form of a vial, ampule, tube, blister pack, cartridge or capsule.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the pharmaceutical composition.

Even more preferably a freeze-dried preparation of one or more pro-regenerative factor, preferably a TFF member, more preferably TFF3 may be pre-packaged for example in single dose units. In an even more preferred embodiment the single dose unit is adjusted to the patient.

### REFERENCES

Hoffmann et al., Histol Histopathol 16(I):319-34, 2001
Taupin and Podolsky, Nat Rev Mol Cell Bio. 4(9):721-32, 2003
Thim, Cell Mol Life Sci 53(II-12):888-903, 1997

### EXAMPLES

### Examples

### Example 1

Protocol for Local Pulmonary Treatment with TFF3 Through Bronchoalveolar Lavage (BAL)

### I. Patient Group to be Treated:

Heavy smoking patients with Chronic obstructive pulmonary disease

### II. Treatment Regime:

Inhalation of 2×20 mg TFF3 dry powder.

### III. Analysis of Results:

a) Monitoring of a series of chest x-ray and/or thoracic CT-scan combined with signs and symptoms of pulmonary dysfunction, e.g. dyspnea at rest or after exercise, reduced pulmonary function using forced expiratory flow in first second (FEV1) and measurements of vital capacity (VC); oxygenation capacity as by monitoring the PaO₂/FiO₂ ratio (arterial oxygen tension in mmHg over inspired oxygen fraction). The mucociliary function is tested with inhaled insoluble radioactive particles and subsequent measurements of muciliary clearance rate using scintigraphic measurements of clearance rate.

A successful treatment ciliary clearance rate, limitation of the declining or even improved lung function measurements, e.g. reflected in increased FEV1 and VC and/or reduced signs and symptoms of pulmonary dysfunction i.e. disappearance of dyspnea, reduced dyspnoea, sputum and coughing rate, eventually combined with an improved SAT O₂ and normalized arterial measured using pulse oximetry, provided that the first SAT O₂% was abnormally low and normalized paCO2 (arterial CO2 pressure (arterial puncture)) and in case the pretreatment pH was below 7.4 a pH increase.

Table Overview of monitoration of pulmonary
infections and or pulmonary colonization

| Variable | Signs and symptoms and markers | Before TFF* | TFF3 Reponse |
|---|---|---|---|
| Clinically | Fever, cough, dypnea, purulent sputum, | ↑ | ↓ |
| | quantitative sputum production | | |
| Biomarkers | Inflammatory markers | ↑ | ↓ |
| | PCT, CRP, ESR | | |
| Chest x-ray | Infiltrates, isolated or diffuse | ↑ | ↓ |
| Pulmonary | PaO2/FiO2 ratio | ↓ | ↑ |
| dysfunction | PaCO2 | ↑ | ↓ |
| | Arterial pH | ↓ | pH ~7.4 |
| | Oxygen supplementation of FiO2 | ↑ | ↓ |
| | Need for non-invasive or invasive | ↑ | ↓ |
| | mechanical ventilation | | |
| | Measurements of FEV1, VC, and forced | ↓↓ | ↑ |
| | VC and function of small airways | | |
| Mucocilary | Scintigrafic documented clearance of | ↓ | ↑ |
| clearance rate | inhaled radioactive particles | | |
| BAL derived | Markers of inflammation | ↑ | ↓ |
| monitoring | Cytokines | | |
| | Cells | | |
| | Alveolar macrophages, neutrophils, | ↑ | ↓ |
| | lymphocytes* | | |
| | Measurements of alveolar enzymes | ↑↑ | ↑ |
| Microbiogical | Surveillance cultures, | ↑ | ↓ |
| monitoring | PCT diagnostics | ↑ | ↓ |

*Signs of lack of ciliae or dysfunction of ciliary mucous ladder
Abbreviations:
CRP~C-reactive protein;
PCT~Procalcitonin test;
ESR~Erythrocyte sedimentation rate;
PCR~Polymerase chain reaction;
PaO2~arterial oxygen pressure e.g. in kPa or mmHg;
pH~arterial pH;
PaCO2~arterial CO2 pressure, e.g. in kPa or mmHg;
FiO2~Inspired oxygen fraction or % of oxygen in inspired air.

1. A composition comprising one or more pro-regenerative factors for use as a medicament in a subject for enhancement of airway epithelial repair and/or regeneration and/or normalization of airway epithelial remodelling in the terminal lung unit (TLU), interalveolar septae, pulmonary interstitium and pulmonary parenchyma.

2. The composition of claim 1, wherein an effective amount of one or more pro-regenerative factors is administered to the subject via intratracheal, intrabronchial, intraalveolar or bronchio-alveolar administration.

3. The composition of claim 1, wherein the pro-regenerative factor is a peptide or protein from the trefoil family factor (TFF) family or a variant thereof.

4. The composition of claim 1, wherein the pro-regenerative factor is a dimer of a trefoil family factor (TFF) family peptide.

5. The composition of claim 1, wherein the pro-regenerative factor is trefoil family factor 3 (TFF3) or a variant, derivative or analogue thereof.

6-7. (canceled)

8. The composition of claim 1, wherein the subject has a chronic airway condition.

9. The composition of claim 1, wherein the subject is suffering from a chronic airway condition is selected from the group consisting of Asthma, Chronic obstructive pulmonary disease (COPD), chronic bronchitis, Cystic fibrosis, Bronchiectasis, Panbronchiolitis, lung cancer, pneumonia, ventilator associated pneumonia (VAP), hospital acquired pneumonia (HAP), community acquired pneumonia (CAP), severe community acquired pneumonia (sCAP) and pneumocystis carinii.

10. The composition of claim 1, wherein the subject is administered a nebulized solution, a nebulized aerosol or inhaled powder or a suspension of one or more pro-regenerative factors.

11. (canceled)

12. The composition of claim 1, wherein the subject is administered a solution of one or more pro-regenerative factors via bronchoalveolar lavage or blind tracheal washing, is administered a pegylated, liposomal or nanoparticle prepared from one or more pro-regenerative factors, or is administered one or more pro-regenerative factors during bronchoscopy.

13-15. (canceled)

16. The composition of claim 1, wherein the subject is administered additional drugs selected from a steroid, facilitating growth factor, beta-2 agonist or other bronchodilator, by systemic administration or by inhalation, before, during or after the administration of one or more pro-regenerative factors.

17-19. (canceled)

20. The composition of claim 1, wherein one or more pro-regenerative factors is administered to a subject in an amount of from 0.1 microgram/kg to about 10 milligram/kg body weight per day.

21. The composition of claim 1, wherein the subject is a mammal.

22. The composition of claim 21, wherein the mammal is a human younger than 12 years of age or older than 12 years of age.

23-28. (canceled)

29. A method for enhancing airway epithelial repair and/or regeneration and/or for the normalization of airway epithelial remodeling in a human subject comprising administering an effective amount of one or more pro-regenerative factors to a subject.


SEQUENCE LISTING (3 sequences)

SEQ ID NO: 1 - protein, 84 aa, Homo sapien
MATMENKVICALVLVSMLALGTLAEAQTETCTVAPRERQNCGFPGVTPSQCANKGCCFDDTVRGVPWCFYPNTIDVPPEEECEF

SEQ ID NO: 2 - protein, 129 aa, Homo sapien
MGRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 3 - protein, 80 aa, Homo sapien
MAARALCMLGLVLALLSSSSAEEYVGLSANQCAVPAKDRVDCGYPHVTPKECNNRGCCFDSRIPGVPWCFKPLQEAECTF